(19) United States
(12) Patent Application Publication
(10) Pub. No.: US 2009/0226543 A1
(43) Pub. Date: Sep. 10, 2009

(54) AGENT FOR THE THERAPY AND PROPHYLAXIS OF DIABETES MELLITUS

(75) Inventors: Peter Bendzko, Berlin (DE); Jorg Schulz, Berlin (DE); Klaus Gublin, Crivitz (DE)

Correspondence Address: NORRIS, MCLAUGHLIN & MARCUS, P.A., 875 THIRD AVE, 18TH FLOOR, NEW YORK, NY, 10022, US

(73) Assignee: GM GESUNDHEITSMANAGEMENT, BERLIN (DE)

(21) Appl. No.: 12/089,487

(22) PCT Filed: Oct. 9, 2006
(86) PCT No.: PCT/DE06/01782

§ 371 Date: Sep. 19, 2008

(30) Foreign Application Priority Data
Oct. 7, 2005 (DE) 10 2005 048 483.2

(51) Int. Cl. A61K 33/06

(52) U.S. Cl. 424/684

(57) ABSTRACT

The invention relates to a means of therapy and prophylaxis of Diabetes mellitus Type 1 and Type 2.

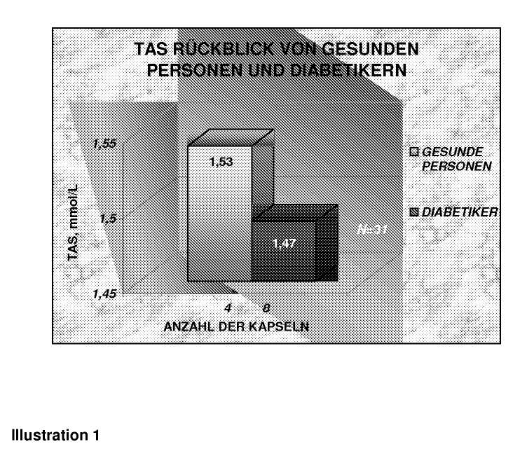

Fields of application of the invention are medicine and the pharmaceutical industry.

The invention has the objective of developing a new kind of means for therapy and prophylaxis of Diabetes mellitus.

The means according to the invention entails physically and/or chemically activated minerals and, if applicable, further substances. Preferred substances are heulandite/klinoptilolith, natrolith or thomsonite. The particle diameter preferably below 5 μm.

The invention relates to a means of therapy (and prophylaxis) of Diabetes mellitus. Fields of application of the invention are medicine and the pharmaceutical industry.

### INTRODUCTION

Diabetes mellitus is caused by an absolute or relative lack of insulin. Inter alia, it leads to an increase of plasma glucose concentration. Depending on the cause and the course, various types of Diabetes mellitus are distinguished.

In Type I Diabetes (insulin-dependent Diabetes mellitus), there is an absolute lack of insulin. The cause is a lesion of the B cells in the pancreatic islets, mainly caused by an autoimmune disease, which can be triggered by a virus infection.

Examinations in animal experiments indicate that free radicals lead to the lesion of the B cells. If the pancreatic islets are inflamed, free oxygen radicals are released in toxic quantities by the infiltrated macrophages and endothelial cells. The B cells of the pancreas are destroyed by free oxygen radicals, as they only have insufficient defense against free radicals.

Type II Diabetes (insulin-independent Diabetes mellitus) is the much more frequent form of diabetes. In Germany, there are currently about 200,000 Type I diabetics, on the other hand about 4 million type II diabetics (MICHAELIS, 1985). In this case, there is a relative lack of insulin. The insulin production can be normal or increased, but the target organs always manifest a reduced sensitivity towards insulin. Patients with type II diabetes are mainly overweight. Adipositas is a consequence of genetic disposition, excessive supply of nutrition and too little movement.

Type II diabetics suffer from hypertonia and metabolic disorders much more frequently than non-diabetics (also called Metabolic Syndrome, REAVEN, 1991). Up to now, type II diabetes has mainly affected older people (“old-age diabetes”)—for some years now, there has been an alarming increase of incidence in young people in the general population (annual conference of the American Diabetes Association, San Diego, 1999).

As a result of these metabolic lapses, there are finally macro- and micro-circulation disorders with the typical organic manifestations. The consequences are then in particular damage to the eyes, renal diseases, ischemic diseases of the heart and peripheral vascular system as well as the occurrence of cerebral and neurological damage.

Examinations on animals indicate that free radicals support destruction of the cellular islets in the pancreas in the pathogenesis of the Diabetes mellitus. Reactive intermediate oxygen products, released in toxic quantities by endothelial cells and infiltrated macrophages in the course of the inflammation of the beta-cell tissue in the pancreas, lead to massive damage and destruction of beta cells. Numerous examinations indicate that a diabetic condition is connected with oxidative stress.

Oxidative stress is also a synonym for diabetes. In this context, it is responsible for the following complications: polyneuropathy, retinopathy and angiopathy. Although hundreds of papers on the significance of free radicals and application of antioxidants in diabetes have been published, antioxidants have still not been included in the official protocols of anti-diabetes therapy.

Much development research work on experimental animals has confirmed the protective effect of components which remove radicals in the cellular islets (e.g. nicotinamide). Markers for oxidative stress are seen in diabetes in particular when the amount of Vitamin C has been reduced.

As protection against free radicals, the organism has an antioxidative protective system, which develops the antioxidants. The most important antioxidants are the antioxidative enzymes glutathione peroxidase and superoxide dismutase. Their essential components are selenium, zinc, copper and manganese as well as non-enzymatic antioxidants such as alpha-tocopherol (Vitamin E), beta-carotene (Provitamin A), ascorbic acid (Vitamin C), melatonin and glutathion and many more besides.

Antioxidative protective agents mutually influence one another. For example, Vitamin E (which occurs in the blood as D-alpha-tocopherol) is “regenerated” by Vitamin C (ascorbic acid).

Fat-soluble Vitamin E located in the cell membrane is regarded as the first line of defense against peroxidation. As a radical trapper, it ends chain reactions in the cell membrane and thus limits cell membrane damage to distinct areas.

Water-soluble Vitamin C (ascorbic acid) not only protects the cell membrane by Vitamin E regeneration, but also the interior of the cell, in particular the cell core, by acting as a redox catalyst in the cytoplasm of the cell and “neutralising” free radicals. In this context, the ascorbic acid transfers into its oxidation product, dehydroascorbic acid, by giving off hydrogen. It changes the cell structure and can lead to neurotoxic effects. This reaction is reversible in a normal cell.

The damaged metabolism of ascorbic acid (Vitamin C) is also connected with diabetes. Ascorbate is needed in vivo for regeneration of Vitamin E and can be oxidised to form dehydroascorbic acid, which can change the cell structure and have a neurotoxic effect. In healthy tissue, the dehydroascorbic acid converts back into ascorbic acid. The increased origination of large quantities of dehydroascorbic acid leads to a higher sensitivity of the cells against oxidation damage. Other examinations show that a high level of glucose in diabetes is connected with the introduction of ascorbic acid, which results in a low level of ascorbate in the cells.

The main question to be asked in evidently increased oxidation stress is whether the various indices indicating the effect of free radicals are a sign of oxidation stress as an aetiological factor or whether they have been generated secondarily as a result of the damaged tissue. In addition, the evidence of primary oxidation stress can be a result of the examination of antioxidant reserves and from the improvement of the status in the addition of antioxidants. In connection with this, it must be stated that the level of ascorbic acid in the plasma is certainly lower with diabetics, both with human and also with animal groups, whereas the dehydroascorbate, primarily an oxidation product, is increased. On the other hand, the level of ascorbic acid in the single-nuclear leukocytes of patients with diabetes is reduced.

The life expectancy of a 40-year-old with newly diagnosed diabetes is reduced by 8 years on average. 75% of all type 2 diabetics (or 35% of all type 1 diabetics) die of cardiovascular complications. In comparison with persons without diabetes, the risk of CHD (coronary heart disease) in type 2 diabetics is increased by more than threefold, that of a cerebro-vascular disease by more than double.

To sum up, it can be stated that there is still no satisfactory treatment method for diabetes diseases and that thus a health policy problem of the first order is still waiting for a solution.

The invention has the objective of developing a new kind of means for therapy and prophylaxis of diabetes.

This objective is achieved by the means described in Claim 1. Surprisingly, it was seen that a means entailing physically and chemically modified minerals, in particular minerals containing silicon such as zeoliths or bentonite, can be suitable in combination with further substances such as extracts of plants of the Urticaceae species for therapy and prophylaxis of Diabetes mellitus. The means can be used for both type I and also type II diabetes.

In the invention, both natural and also synthetic zeoliths are used, preferably one or more of the following kinds of zeolith: heulandite/klinoptilolith, natrolith or thomsonite. These kinds of zeolith contain about 20-30% magnesium and calcium shares. A specific preference is micronised heulandite/klinoptilolith with a particle diameter of less than 5 μm.

The components of the means according to the invention are contained in the following quantities:

Mineral 70-98% and extracts of plants of the Urticaceae species 2-30%, preferably zeolith 75% and stinging nettle extract 25%.

The means is used as a rule in the form of a nutrition supplement, if applicable in combination with further substances. The preferred form is oral consumption in the form of powder, tablets or capsules.

The following clinical results have been achieved with the means according to the invention:

Activated klinoptilolith in combination with extracts from plants of the Urticaceae species develops a strong antioxidative effect, about 20 times stronger than Vitamin C or Vitamin E. This is why consumption of activated klinoptilolith in combination with extracts of plants of the Urticaceae species leads to a greatly increased antioxidative capacity of the organism.

In patients with diabetes mellitus, who took activated klinoptilolith with extracts of plants of the Urticaceae species, the activities of the endogenous antioxidative enzymes SOD, glutathione peroxidase (GPx) and glutathione reductase (GR) were determined with the help of the commercially available reagent ABTS from the firm of Randox.

In healthy persons taking 4 capsules of activated klinoptilolith with stinging nettle extract a day, the mean TAS value of 1.55 mmol/l was unambiguously higher in comparison with the persons who did not take activated klinoptilolith and stinging nettle extract.

All examinations up to now indicate that hyperglycaemia results in oxidative stress with a weakening of the antioxidative protective system. The results are damage to the peripheral neurons and vessels, leading to the development of late complications of Diabetes mellitus such as polyneuropathy, microangiopathy, in particular retinopathy and glomerulosclerosis. These complications can be prevented to a great extent by increasing the endogenous antioxidative potential by taking exogenous antioxidants, thus also reducing the negative effect of the oxidative stress.

It is conspicuous that primarily patients with insulin-independent diabetes reacted to the taking of activated klinoptilolith with stinging nettle extract with its antioxidative effect.

Further, the invention relates to the fact that diabetics of type 1 and type 2 profit from the fact that the cell is put into a relative oxygen debt. This can be achieved by a physical movement programme at altitude, in altitude training centres under normobar hypoxia or with a changed respiratory quotient (RQ smaller or equal 0.85) as a result of certain changes in the air in inhalation (changed oxygen and nitrogen concentration). By administration of activated zeoliths, also in combination with extracts of plants of the Urticaceae species and Omega-3 fatty acids, a considerable improvement of the overall image of the metabolic syndrome and in particular of Diabetes mellitus can be achieved. As expected, the state of health of diabetics can be considerably increased by the use of the present invention.

The invention is to be explained below on the basis of embodiments.

### EMBODIMENT 1

### Clinical Case Control Study with Activated Klinoptilolith with Stinging Nettle Extract in Patients with Type 2 Diabetes Mellitus

As a result of the positive examination results both in animal experiments and also in the clinical area, there was a clinical case control study on 30 patients with secured type 2 Diabetes mellitus already in existence for years under ambulant treatment and control conditions.

### Methodical Procedure

30 patients with type 2 Diabetes mellitus were given 3×3 capsules of activated klinoptilolith with stinging nettle extract every day for 3 months. Control examinations were held at the start of the case observation study and after 6 and 12 weeks.

The following were examined by laboratory chemistry:
C-peptide
Insulin
Proinsulin
HbA₁c
Cholesterol
HDL cholesterol
LDL cholesterol
Triglyceride
Blood sugar

All the examinations (including 1^{st} and 2^{nd} control) were done within 2 consultations by a general practitioner and were monitored medically.

### Results

In 30 patients with type 2 Diabetes mellitus already in existence for years, it was seen after 3 months of treatment with activated klinoptilolith with stinging nettle extract that the C-peptide, HbA and the triglycerides had developed in a significantly positive way. These results permit conclusions above new therapy variants for Diabetes mellitus. In strongly overweight patients

TABLE 1
Mean values (MV) and standard deviation (SD) of laboratory figures
according to Diabetes type at the individual times of examination

| | Diabetes | | 1^{st} examination | | 2^{nd} examination | | 3^{rd} examination | |
|---|---|---|---|---|---|---|---|---|
| Lab figures | Type | Quantity | MW | SD | MW | SD | MW | SD |
| C-Peptide | Dietetic | 8 | 3.89 | 2.65 | 4.54 | 2.57 | 5.13 | 1.92 |
| | Tablets | 12 | 3.77 | 1.79 | 3.96 | 2.14 | 5.43 | 2.39 |
| | Insulin | 10 | 2.51 | 1.83 | 3.11 | 2.02 | 3.00 | 1.06 |
| | Total | 30 | 3.38 | 2.08 | 3.83 | 2.22 | 4.54 | 2.15 |
| Insulin | Dietetic | 8 | 17.31 | 10.92 | 33.28 | 32.95 | 22.90 | 13.17 |
| | Tablets | 12 | 21.08 | 16.73 | 29.49 | 27.97 | 30.75 | 27.69 |
| | Insulin | 10 | 41.63 | 23.55 | 74.58 | 46.48 | 51.65 | 40.38 |
| | Total | 30 | 26.92 | 20.53 | 45.53 | 40.84 | 35.62 | 31.33 |
| Proinsulin | Dietetic | 8 | 39.58 | 23.14 | 32.90 | 21.84 | 34.71 | 25.53 |
| | Tablets | 12 | 24.08 | 19.69 | 25.88 | 20.84 | 23.13 | 20.48 |
| | Insulin | 10 | 22.04 | 14.07 | 18.14 | 14.02 | 14.58 | 8.96 |
| | Total | 30 | 27.52 | 19.82 | 25.17 | 19.35 | 23.37 | 20.09 |
| HbA1C | Dietetic | 8 | 6.61 | 0.65 | 6.65 | 0.80 | 6.85 | 0.92 |
| | Tablets | 12 | 7.13 | 1.20 | 7.05 | 1.21 | 7.11 | 0.93 |
| | Insulin | 10 | 6.85 | 0.60 | 6.63 | 0.70 | 6.86 | 0.67 |
| | Total | 30 | 6.90 | 0.90 | 6.80 | 0.95 | 6.96 | 0.83 |
| Cholesterol | Dietetic | 8 | 6.34 | 1.59 | 6.26 | 1.31 | 6.08 | 0.91 |
| | Tablets | 12 | 5.60 | 1.38 | 5.95 | 1.51 | 5.73 | 1.61 |
| | Insulin | 10 | 4.93 | 0.70 | 5.02 | 0.72 | 4.83 | 0.66 |
| | Total | 30 | 5.57 | 1.34 | 5.72 | 1.31 | 5.52 | 1.26 |
| HDL | Dietetic | 8 | 1.33 | 0.32 | 1.46 | 0.32 | 1.33 | 0.32 |
| Cholesterol | Tablets | 12 | 1.31 | 0.29 | 1.37 | 0.31 | 1.36 | 0.33 |
| | Insulin | 10 | 1.21 | 0.24 | 1.19 | 0.23 | 1.22 | 0.25 |
| | Total | 30 | 1.28 | 0.28 | 1.33 | 0.30 | 1.30 | 0.30 |
| LDL | Dietetic | 7 | 3.93 | 1.35 | 3.72 | 1.19 | 3.36 | 0.70 |
| Cholesterol | Tablets | 11 | 3.59 | 1.16 | 3.73 | 1.18 | 3.47 | 1.23 |
| | Insulin | 9 | 3.11 | 0.69 | 3.10 | 0.69 | 2.94 | 0.69 |
| | Total | 27 | 3.52 | 1.09 | 3.50 | 1.04 | 3.26 | 0.97 |
| Triglycerides | Dietetic | 8 | 2.74 | 1.58 | 3.05 | 1.81 | 3.49 | 2.46 |
| | Tablets | 12 | 1.79 | 1.34 | 1.86 | 0.82 | 2.28 | 1.48 |
| | Insulin | 10 | 1.65 | 1.60 | 1.60 | 0.78 | 1.75 | 1.21 |
| | Total | 30 | 1.99 | 1.51 | 2.09 | 1.26 | 2.42 | 1.80 |
| Blood sugar | Dietetic | 8 | 7.32 | 2.18 | 8.44 | 2.39 | 8.61 | 2.22 |
| | Tablets | 12 | 10.16 | 4.64 | 9.91 | 3.45 | 11.25 | 3.97 |
| | Insulin | 10 | 9.13 | 4.78 | 9.50 | 4.53 | 9.59 | 4.19 |
| | Total | 30 | 9.06 | 4.21 | 9.38 | 3.55 | 9.99 | 3.72 |

(BROCA Index >18), the effectivity of 3 × 3 capsules of activated klinoptilolith with stinging nettle extract was less easy to detect: here, the dosage is to be varied as a function of the body weight.
The most important data can be seen from the following tables.

TABLE 2
Mean values (MV) and standard deviation (SD) of the laboratory figures
according to Broca Index groups at the individual times of examination.
In this context, “slightly increased” means up to 18 kg overweight,
“increased” 18 kg and more overweight.

| | Broca | | 1^{st} examination | | 2^{nd} examination | | 3^{rd} examination | |
|---|---|---|---|---|---|---|---|---|
| Lab figures | Index | Quantity | MW | SD | MW | SD | MW | SD |
| C-Peptide | normal | 8 | 3.10 | 1.74 | 3.20 | 2.39 | 3.81 | 2.01 |
| | slightly | 13 | 2.76 | 2.18 | 3.59 | 2.33 | 4.51 | 2.56 |
| | increased | | | | | | | |
| | increased | 9 | 4.53 | 1.93 | 4.74 | 1.81 | 5.24 | 1.53 |
| | total | 30 | 3.38 | 2.08 | 3.83 | 2.22 | 4.54 | 2.15 |
| Insulin | normal | 8 | 23.84 | 18.91 | 45.78 | 48.77 | 31.36 | 23.73 |
| | slightly | 13 | 27.88 | 25.10 | 44.39 | 46.88 | 38.48 | 38.55 |
| | increased | | | | | | | |
| | increased | 9 | 28.29 | 16.00 | 46.96 | 25.78 | 35.29 | 28.38 |
| | total | 30 | 26.92 | 20.53 | 45.53 | 40.84 | 35.62 | 31.33 |
| Proinsulin | normal | 8 | 25.35 | 23.66 | 28.65 | 24.09 | 16.25 | 10.81 |
| | slightly | 13 | 25.18 | 19.53 | 18.16 | 13.89 | 16.57 | 8.53 |
| | increased | | | | | | | |
| | increased | 9 | 32.82 | 17.80 | 32.20 | 20.32 | 39.52 | 28.85 |
| | total | 30 | 27.52 | 19.82 | 25.17 | 19.35 | 23.37 | 20.09 |
| HbA1C | normal | 8 | 6.55 | 0.61 | 6.31 | 0.78 | 6.61 | 0.61 |
| | slightly | 13 | 6.91 | 1.13 | 6.89 | 1.12 | 6.95 | 0.91 |
| | increased | | | | | | | |
| | increased | 9 | 7.19 | 0.69 | 7.11 | 0.75 | 7.28 | 0.82 |
| | total | 30 | 6.90 | 0.90 | 6.80 | 0.95 | 6.96 | 0.83 |
| Cholesterol | normal | 8 | 6.22 | 1.97 | 6.59 | 1.43 | 6.15 | 1.34 |
| | slightly | 13 | 5.11 | 0.70 | 5.15 | 0.83 | 4.92 | 0.93 |
| | increased | | | | | | | |
| | increased | 9 | 5.66 | 1.27 | 5.78 | 1.45 | 5.84 | 1.33 |
| | total | 30 | 5.57 | 1.34 | 5.72 | 1.31 | 5.52 | 1.26 |
| HDL | normal | 8 | 1.41 | 0.32 | 1.50 | 0.41 | 1.48 | 0.32 |
| Cholesterol | slightly | 13 | 1.27 | 0.27 | 1.26 | 0.25 | 1.25 | 0.29 |
| | increased | | | | | | | |
| | increased | 9 | 1.20 | 0.23 | 1.29 | 0.20 | 1.23 | 0.26 |
| | total | 30 | 1.28 | 0.28 | 1.33 | 0.30 | 1.30 | 0.30 |
| LDL | normal | 8 | 4.15 | 1.49 | 4.24 | 1.05 | 3.83 | 1.08 |
| Cholesterol | slightly | 11 | 3.09 | 0.45 | 3.11 | 0.70 | 2.86 | 0.72 |
| | increased | | | | | | | |
| | increased | 8 | 3.48 | 1.07 | 3.40 | 1.23 | 3.31 | 1.02 |
| | total | 27 | 3.52 | 1.09 | 3.50 | 1.04 | 3.26 | 0.97 |
| Triglycerides | normal | 8 | 1.47 | 0.99 | 1.83 | 0.99 | 1.82 | 1.26 |
| | slightly | 13 | 2.05 | 1.77 | 1.73 | 0.91 | 2.12 | 1.54 |
| | increased | | | | | | | |
| | increased | 9 | 2.39 | 1.51 | 2.85 | 1.66 | 3.41 | 2.26 |
| | total | 30 | 1.99 | 1.52 | 2.09 | 1.26 | 2.42 | 1.80 |
| Blood sugar | normal | 8 | 8.92 | 4.45 | 8.62 | 4.06 | 8.85 | 2.61 |
| | slightly | 13 | 8.69 | 4.11 | 9.67 | 4.15 | 10.77 | 4.78 |
| | increased | | | | | | | |
| | increased | 9 | 9.71 | 4.56 | 9.65 | 2.18 | 9.89 | 2.76 |
| | total | 30 | 9.06 | 4.21 | 9.38 | 3.55 | 9.99 | 3.72 |

### EMBODIMENT 2

### Clinical Experience with Type 2 Diabetes Mellitus Patients in the Combination of Altitude Training (Normobar Hypoxia) with Administration of Activated Zeolith Combined with Extract of Plants of the Urticaceae Species and Omega-3 Fatty Acids

8 patients were subjected to 30-minute treadmill training at a simulated altitude of 1,500 m for three weeks. In addition, these patients were given 1000 mg Omega-3 fatty acids and minerals containing silicon (activated klinoptilolith/stinging nettle extract, 3×2 capsules, corresponding to 6 grams) per day. Before and after, the following parameters were determined by laboratory chemistry: C-peptide, Insulin, Proinsulin, HbA₁C, Cholesterol, HDL cholesterol, LDL cholesterol, triglycerides, blood sugar, insulin requirement, body weight.

In this context, the following changes were observed: C-peptide—unchanged, Insulin—unchanged, Proinsulin—slightly reduced, HbA₁C—slightly reduced, cholesterol—clearly reduced, HDL cholesterol—unchanged, LDL cholesterol—clearly reduced, triglycerides—slightly reduced, blood sugar—clearly reduced, Insulin requirement—very clearly reduced, body weight—very clearly reduced.

From the results obtained, a clinical relevance in the sense of a positive influence of the metabolic syndrome can be recognised.

### Description of Illustration:

Illustration 1 shows the values of the Total Antioxidant Status (TAS) of diabetics with a TAS of 1.28 mmol/L before taking activated klinoptilolith with stinging nettle extract and developed a TAS value of 1.53-1.47 mmol/l following one month of taking an average of 4-8 capsules of activated klinoptilolith with stinging nettle extract per day (2-4 grams total dose).

1. Means for therapy and prophylaxis of Type 1 and Type 2 Diabetes mellitus, entailing physically and/or chemically activated minerals and, if applicable, further substances.

2. Means according to claim 1, in which the physically and/or chemically activated minerals are minerals containing silicon.

3. Means according to claim 1, in which natural or synthetic zeoliths are used as physically and/or chemically activated minerals.

4. Means according to claim 1, in which heulandite/klinoptilolith, natrolith or thomsonite are used as natural zeoliths.

5. Means according to claim 4, wherein micronised heulandite/klinoptilolith with a particle diameter of less than 5 μm is used.

6. Means according to claim 3, in which NaY zeolith is used as a synthetic zeoliths.

7. Means according to claim 1, in which bentonite is used as a physically and/or chemically activated mineral.

8. Means according to claim 1, in which extracts of plants of the Urticaceae species are added as further substances.

9. Means according to claim 1, containing the following components in the following quantities:
Physically and/or chemically activated minerals 70-98%
Extracts of plants of the Urticaceae species 2-30%.

10. Means according to claim 9, containing the following components in the following quantities:

| | |
|---|---|
| Physically and/or chemically activated minerals | 75% |
| Extracts of plants of the Urticaceae species | 25% |

11. A nutrition supplement comprising the means of claim 1, optionally in combination with further substances, preferably unsaturated fatty acids such as Omega-3 fatty acids.

12. A nutrition supplement comprising the means of claim 1 for use under conditions of normobar hypoxia or of a defined respirator coefficient of smaller or equal 0.85.

13. (canceled)

14. (canceled)
